(19) United States
(12) Patent Application Publication
(10) Pub. No.: US 2006/0058772 A1
(43) Pub. Date: Mar. 16, 2006

(54) Absorbent article having a loopless fastening system

(76) Inventor: Hamzeh Karami, Brewster, MA (US)

Correspondence Address: AMSTER, ROTHSTEIN & EBENSTEIN LLP, 90 PARK AVENUE, NEW YORK, NY, 10016, US

(21) Appl. No.: 10/938,685

(22) Filed: Sep. 10, 2004

(51) Int. Cl. A61F 13/15

(52) U.S. Cl. 604/391; 604/392

(57) ABSTRACT

An absorbent article including a topsheet, a backsheet, and an absorbent core sandwiched between the topsheet and the backsheet, a back waist portion having an inner surface, an outer surface, a first lateral end and a second lateral end, and a front waist portion having an inner surface, an outer surface, a first lateral end and a second lateral end. At least one hook-type fastener is disposed at each of the first and second lateral ends of the back waist portion. A crotch portion longitudinally connects the back and front waist portions. At least a portion of the outer surface of the front waist portion is made of a bonded nonwoven subjected to at least one of a water jet treatment and aperturing, and the at least one hook-type fastener is releasably attachable to the outer surface of the front waist portion.

### TECHNICAL FIELD

The present disclosure relates to an absorbent article, and more particularly to an absorbent article having a fastening system that does not require a conventional loop-type fastening element.

### BACKGROUND OF THE INVENTION

Since the advent of VELCRO fastener assemblies wherein a hook-type fastener element composed of a plurality of minihooks (very small hooks) engages a loop-type fastener element composed of a multitude of miniloops (very small loops), their use has become more or less ubiquitous in the field of disposable absorbent articles (such as diapers, T-shape briefs and preformed pull-ups) due to their convenience, reliability and inexpensiveness. A primary limitation on their widespread use has been the need for each minihook fastener element to have a corresponding miniloop fastener element to engage with—i.e., to serve as a loop-type “landing zone” for the minihook fastener element.

Thus, instead of an article or article portion containing a minihook fastener element capable of engaging anywhere along a facing surface of another article or article portion, it has been necessary for the two articles or article portions to be designed such that, during use, the minihook and miniloop fastener elements will be appropriately positioned opposite one another (i.e., juxtaposed) for engagement with one another. This disadvantage of the fastener assembly has been ameliorated to some degree by the use of minihooks fastener elements of relatively small area adapted to engage miniloop fastener elements of relatively great area so that each minihook fastener element could be positioned as desired anywhere within the area of each miniloop fastener element. (The preference for a small minihook fastener element relative to a large miniloop fastener element, rather than vice versa, is founded on both the relative inexpensiveness and the relatively softer, less irritating nature of the latter). Thus, conventional woven or nonwoven articles have had affixed to the surface thereof along one axis (e.g., sewn thereon) a miniloop fastener element strip of greater longitudinal dimensions then the corresponding minihook fastener element, thereby to provide the user with freedom of choice as to where the minihook fastener element would be positioned along the miniloop fastener element strip. For example, see U.S. Pat. No. 5,383,872, U.S. Pat. No. 5,624,429 and U.S. Pat. No. 5,527,305. In this manner the user is provided with a degree of flexibility or choice along one axis, albeit at additional material and manufacturing costs for the strip. It has even been proposed that one entire surface of an article be constituted of a miniloop fastener element in order to provide the user with total flexibility and choice (that is, the ability to position the minihook fastener element anywhere on the article surface), albeit at an even greater material and manufacturing cost for the surface. See, for example, U.S. Pat. No. 5,019,065.

Disposable absorbent articles of the types mentioned hereinabove have different requirements as to the peel strength and shear strength of the various engagements depending, for example, on whether the engagement is about a waist or elsewhere, whether its fastening power is to be supplemented by the power of additional and possibly different fastener systems, etc. Generally a peel strength of at least 60 grams per square inch and a shear strength of at least 1300 grams per square inch is preferred.

The need to provide a miniloop fastener element—whether as a landing zone, as a strip, or as an entire surface—has stifled the development of uses for the minihook fastener element in various situations where it might prove advantageous—for example, to provide size adaptability (especially to enable an absorbent article to accommodate both persons of normal waist size and persons of smaller than normal waist size), to enable employment in situations where the benefit to be obtained does not economically justify the added material and manufacturing costs of providing both minihook and miniloop fastener elements, etc.

Thus, the need remains for a fastening system for an absorbent article that does not require a loop fastening element to mate with a hook-type fastening element.

Accordingly, an object of the present invention is to provide an absorbent article which, in at least one preferred embodiment, incorporates a fastener assembly including a hook-type fastening element that does not require a corresponding loop-type fastening element.

Another object is to provide a fastener assembly for an absorbent article which, in at least one embodiment, affords enhanced shear and peel strength.

A further object is to provide a fastener assembly for an absorbent article that, in at least one embodiment, allows for easy size adjustability over a wide range of sizes.

### SUMMARY OF THE INVENTION

A fastening system according to an exemplary embodiment of the invention includes at least one hook-type fastener, and at least a portion of a bonded nonwoven surface subjected to at least one of water jet treatment and aperturing. The at least one hook-type fastener is releasably attachable to the portion of the bonded nonwoven surface.

An absorbent article according to an exemplary embodiment of the invention includes a topsheet, a backsheet, and an absorbent core sandwiched between the topsheet and the backsheet, a back waist portion having an inner surface, an outer surface, a first lateral end and a second lateral end, and a front waist portion having an inner surface, an outer surface, a first lateral end and a second lateral end. At least one hook-type fastener is disposed at each of the first and second lateral ends of the back waist portion. A crotch portion longitudinally connects the back and front waist portions. At least a portion of the outer surface of the front waist portion is made of a bonded nonwoven subjected to at least one of a water jet treatment and aperturing, and the at least one hook-type fastener is releasably attachable to the portion of the outer surface of the front waist portion.

An absorbent article according to another exemplary embodiment of the invention includes a topsheet, a backsheet, an absorbent core sandwiched between the topsheet and the backsheet, a back waist portion having an inner surface, an outer surface, a first lateral end and a second lateral end, and a front waist portion having an inner surface, an outer surface, a first lateral end and a second lateral end. A first wing extends from the first lateral end of the back waist portion, and a second wing extends from the second lateral end of the back waist portion. At least one hook-type fastener is disposed at each of the first and second lateral ends of the front waist portion and at a lateral free end of one of the first and second wings. A crotch portion longitudinally connects the back and front waist portions. At least a portion of the outer surface of at least one of the first and second wings is made of a bonded nonwoven subjected to at least one of a water jet treatment and aperturing. The hook-type fasteners are releasably attachable to the bonded nonwoven.

In at least one embodiment of the invention, the bonded nonwoven is subjected to a water jet treatment and has a pattern of three-dimensional relief structures.

In at least one other embodiment of the invention, the bonded nonwoven is subjected to a water jet treatment, has a pattern of three-dimensional relief structures and is apertured.

In various exemplary embodiments of the present invention, the absorbent article can be a T-shaped or a conventionally-shaped diaper or a pull-on undergarment.

### BRIEF DESCRIPTION OF THE DRAWING

The above and related objects, features and advantages of the present invention will be more fully understood by reference to the following detailed description of the presently preferred, albeit illustrative, embodiments of the present invention when taken in conjunction with the accompanying drawing wherein:

FIG. 1 is a plan view of the inside surface of a stretched out T-shaped diaper according to an exemplary embodiment of the invention;

FIG. 2 is a cross-sectional view along the line A-A′ of FIG. 1;

FIG. 3 shows the T-shaped diaper of FIG. 1 with wing portions overlapped to form a belt;

FIG. 4 shows a cross-sectional view of a bonded nonwoven having three-dimensional relief structures according to an exemplary embodiment of the invention;

FIG. 5 is a plan view of the inside surface a stretched out T-shaped diaper according to another exemplary embodiment of the invention;

FIG. 6 is a partially cut away plan view of the inside surface of a stretched out disposable diaper according to an exemplary embodiment of the invention;

FIG. 7 is a partially cut away plan view of the inside surface of a stretched out pull-up absorbent article according to an exemplary embodiment of the invention; and

FIG. 8 shows a disposable diaper according to another exemplary embodiment of the invention.

### DETAILED DESCRIPTION OF PREFERRED EMBODIMENTS

In various exemplary embodiments of the present invention, an absorbent article is provided with a fastening system that does not include a loop fastening element. Instead, hook-type fasteners are able to securely attach to a bonded nonwoven surface that has been subjected to a water jet treatment and/or aperturing. The nonwoven can also be provided with a pattern of three-dimensional relief structures during the water jet treatment, resulting in even greater shear and peel strength. The absorbent article can be a T-shaped or conventional shaped diaper, a pull-up undergarment, a breathable diaper or any other garment that incorporates a fastening system.

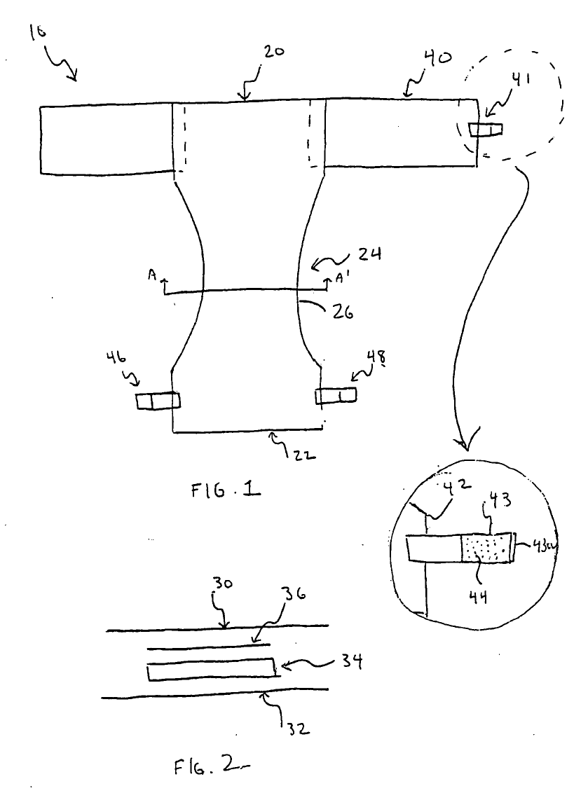

FIG. 1 is a plan view of the T-shaped diaper according to an exemplary embodiment of the invention. As used herein, the term “diaper” generally refers to an absorbent article worn about the torso of an infant or incontinent person. As shown in FIG. 1, the T-shaped diaper 10 includes a back portion 20, a front portion 22 and a crotch portion 24 that connects the back portion 20 to the front portion 22. As is conventional in such constructions, the crotch portion 24, either alone or in combination with one or both of the back and front portions 20, 22, defines leg openings 26 adapted to snugly receive the wearer's legs. The leg openings 26 may be elasticized for a better, more leakproof fit with the wearer's legs.

As shown in FIG. 2, which is a cross-sectional view of the T-shaped diaper 10 along the line A-A′, the T-shaped diaper 10 also includes a topsheet 30 (also referred to as a body-facing sheet, liner or coversheet) and a backsheet 32 (also referred to as a garment-facing sheet or barrier sheet). An absorbent core 34 is disposed intermediate the topsheet 30 and backsheet 32. Optionally, an acquisition/distribution layer 36 is disposed intermediate the absorbent core 34 and the topsheet 30.

Topsheet 30 may be made of any suitable relatively liquid-pervious material currently known in the art or later discovered that permits passage of a liquid therethrough. Examples of suitable top sheet materials include spun-bonded or carded nonwoven webs of polypropylene, polyethylene, nylon, polyester and blends of these materials, perforated, apertured or reticulated films, and the like. Nonwoven materials are exemplary because such materials readily allow the passage of liquids to the underlying acquisition layer 36, and therethrough to absorbent core 34. The top sheet 30 is preferably formed of a single ply of nonwoven material that may be made of thermally bonded, spunbonded fibers, a spunbond-meltblown-spunbond web, or fibers that have been hydroentangled, having a basis weight of 10-30 grams per square meter and having appropriate strength and softness for use as a topsheet in an application which will be in contact with human skin. Topsheet 30 may be treated with surfactant, rendering it hydrophilic to facilitate the passage of moisture through topsheet 30. The present invention is not intended to be limited to any particular material for top sheet 24 and other top sheet materials will be readily apparent to those skilled in the art.

Acquisition layer 36 may be a single layer or multiple layers made of synthetic or natural material, or a combination of both, or a single multilayer apertured film. Acquisition layer 36 serves to quickly collect and distribute discharged body fluid to absorbent core 34. Because such fluid is typically discharged in gushes, the area of absorbent core 34 proximate to the point of fluid discharge may be overwhelmed by the discharge rate, resulting in a leak. Therefore, the acquisition layer 36 facilitates transport of the fluid from the point of discharge across its surface area to contact other parts of absorbent core 34 into which it can be more readily absorbed. The use of an acquisition layer is well known in the art. Accordingly, acquisition layer 36 of diaper 10 of the present invention may have any well known or as yet undiscovered composition.

Absorbent core 34 may be any absorbent material which is generally compressible, conformable to the shape of the wearer's body and not impeding normal movement by the wearer, and capable of absorbing and retaining liquids such as urine and certain other body exudates. The absorbent core 34 may be manufactured in a wide variety of sizes and shapes, (e.g., rectangular, hourglass, “T”-shaped, asymmetric, etc.) and from a wide variety of liquid-absorbent materials commonly used in disposable diapers and other absorbent articles such as wood pulp fluff. Examples of other suitable absorbent materials include creped cellulose wadding; meltblown polymers; chemically stiffened, modified or cross-linked cellulosic fibers; tissue including tissue wraps and tissue laminates; absorbent foams; absorbent sponges; superabsorbent polymers; absorbent gelling materials; or any equivalent material or combinations of materials. The configuration and construction of absorbent core 34 may also be varied (e.g., the absorbent core may have varying caliper zones, a hydrophilic gradient, an absorbent gelling material gradient, or lower average density and lower average basis weight acquisition zones; or may comprise one or more layers or structures, i.e., members, including sheets or webs.) In addition, each member need not be formed of a single unitary piece of material, but may be formed of a number of smaller strips or components joined together lengthwise or width-wise, as long as they are in fluid communication with one another. The total absorbent capacity of absorbent core 34 should, however, be compatible with the design loading and the intended use of the diaper 10. Further, the size and absorbent capacity of the absorbent core 34 may be varied to accommodate wearers ranging from infants through adults.

Backsheet 32 is made of a liquid and/or vapor-impervious material which may be selected from the same group of materials from which the top sheet was selected and preferably having a weight of between 5-45 grams per square meter. Unlike topsheet 30, however, the material used for backsheet 32 is preferably rendered hydrophobic by omitting the surfactant discussed above with respect to topsheet 30. Backsheet 32 may be manufactured by well known methods such as thermal bonding, chemical bonding, spun bonding and hydroentanglement. The backsheet 32 may also be a composite of a film and at least one nonwoven layer attached together by adhesive or heatbonding. The film may be vapor and air permeable but liquid impermeable. The width of the film layer may be narrower than the outer nonwoven layer.

A pair of wings 40 extend laterally from respective lateral edges of the back portion 20 of the T-shaped diaper 10. At least one hook-type fastener 41 is disposed at a free end of one of the wings 40. Hook-type fasteners 46 and 48 are also disposed at respective lateral end portions of the front portion 22. Hook-type fastener 41 includes a first portion 42 permanently attached to the free end of one of the wings 40 and a second portion 43 including hook-type material 44. A finger-lift portion 43a can be provided adjacent second portion 43 at the free end of the hook-type fastener 41. When the T-shaped diaper 10 is in use, the hook-type fastener 41 is unfolded by peeling the second portion 43 from the inner surface of the back portion 20 using the finger lift portion 43a so that the hook-type material 44 extends from the lateral edge of the wing 40 and is oriented towards the body of the wearer. Hook-type fasteners 46 and 48 have the same construction as hook-type fastener 41, and thus will not be discussed herein.

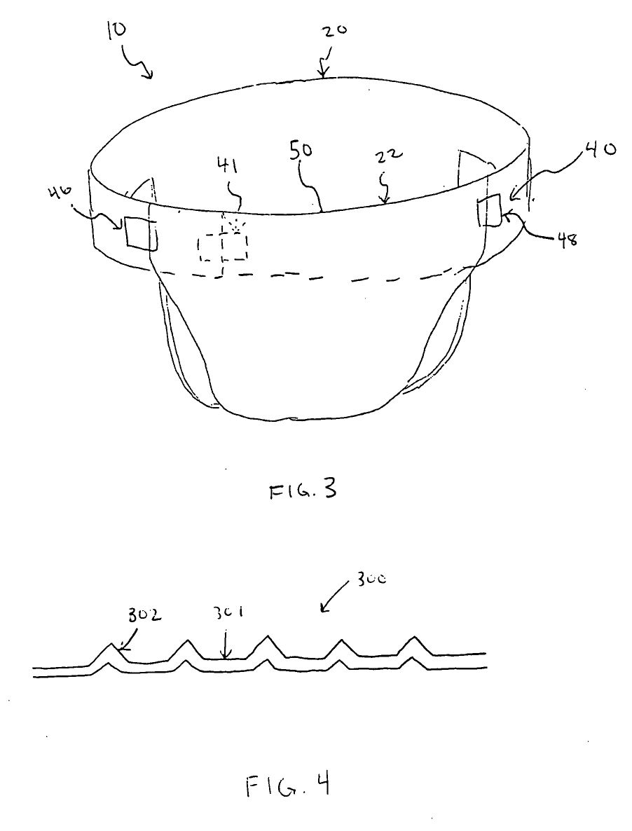

When the T-shaped diaper 10 is first placed on the wearer, the free ends of the two wings 40 are brought around the sides and front of the waist of the wearer and make a partially overlapping connection about the front of the waist of the wearer. Thus, as illustrated in FIG. 3, once the two wings 40 are placed in partially overlapping connection, they, in effect, form (with the back portion 20) a belt 50 encircling the waist of the wearer. The front portion 22 is then passed between the legs of the wearer and raised in front of the wearer until the hook-type fasteners 46 and 48 secured to the front portion 22 engage the exposed outer surface of the belt 44.

The wings 40 may be elastic, non-elastic or partially elastic. Elastic wings may be made of a composite of an elastic film laminated between two nonwovens, preferably spunbonds. The wings 40 may also be made of an elastic nonwoven, made by companies such as: Tredegard of Zurich, Ill. (FABRILEX 307 or 305); Golden Phoenix, which is a subsidiary of BBA Fiber Web of Simpsonville, S.C.; and Advanced Design Concept which is also a subsidiary of BBA Fiber Web.

In the T-shaped diaper according to a preferred embodiment of the present invention, after the nonwoven (that will be used to form the wings 40) is bonded, it is subjected to a water jet treatment. For example, the nonwoven can be bonded by thermal bonding, ultra-sonic bonding, hot-air bonding, chemical bonding, inline hydro-entanglement, mechanical bonding, etc., followed by the water jet treatment. The nonwoven can also be initially asymmetrically bonded (e.g., orthogonally differentially bonded) as per U.S. Pat. Nos. 6,537,644 and 6,610,390, both of which are hereby incorporated by reference.

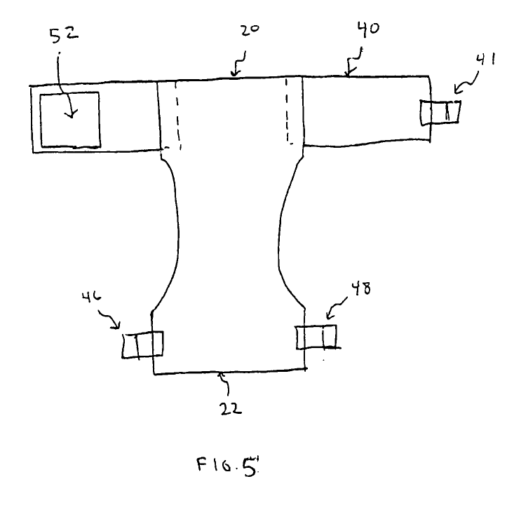

In the preferred water jet treatment, at least one surface of the bonded nonwoven is subjected to at least one row of water jets while an opposite surface of the bonded nonwoven is being sucked by vacuum to a forming surface or screen. The screen may be a simple mesh. The forming surface may have a more elaborate pattern made up of, for example, small squares, circles, rectangular dots or any other suitable shapes so as to create a repeating pattern of three-dimensional relief structures on a surface of the bonded nonwoven. Such a pattern of three-dimensional relief structures formed on a bonded nonwoven is illustrated in FIG. 4. Specifically, FIG. 4 shows a cross-sectional view of a bonded nonwoven 300 having a plurality of three-dimensional relief structures 302 formed on a surface 301 of the bonded nonwoven 300. Although the three-dimensional relief structures 302 shown in FIG. 4 are triangular, as discussed previously, the relief structures may have any suitable shape. The surface of the nonwoven that has the three-dimensional relief structures has better affinity to engage hook-type fasteners as compared to bonded nonwoven without such structures. The water jet machine used in the water jet treatment can be any suitable machine, such as those available from Fleissner GmbH of Germany and Rieter Textile Systems of Switzerland. During the water jet treatment, the applied hydraulic pressure is preferably in the range of about 80 to about 300 bar. Also, preferably about 25 to about 50 holes per inch make up a single row of water jets. The orifice diameter of each water jet is preferably about 0.10 to about 0.15 μm. The traveling speed of the web may be as much as 400 m/min.

Alternatively or in addition, in the T-shaped diaper according to various exemplary embodiments of the invention, the bonded nonwoven used to form the wings 40 can be apertured to formed perforations in the nonwoven. Preferably, the perforations are formed without removing any material from the nonwoven web. This can be done by passing the bonded nonwoven web through a needling machine, in which the web travels between a roll having, for example, between 1 and 1000 pins/per square inch, preferably in the range of about 6 to about 200 pins/sq. in., and an anvil roll having the same number of holes without heating the rolls. As will be discussed in more detail herein, a bonded nonwoven that has perforations has better affinity to engage hook-type fasteners. The pins may have a specific construction such as barbs. Needling machines are available from, for example, Parker Air of Brockton, Mass. In an exemplary aperturing process, the perforations are formed in the nonwoven in line or off line. The aperturing step may take place after the nonwoven feed roll and before a folding station at which the wings are folded. The perforations may be formed on one side of the bonded nonwoven or on both sides of the bonded nonwoven. The bonded nonwoven may be apertured in one step or in more than one step. For example, if the number of perforations is very high, it would be difficult to make a roll having the necessary number of pins. Accordingly, one roll may be used in a first aperturing step having, for example, 300 pins per square inch, in addition to another roll in a second aperturing step having the same number of pins per square inch to form perforations in the nonwoven at a density of 600 perforations per square inch. The water jet treatment and/or aperturing, in effect, provides a softer, bulkier and more flexible nonwoven resulting in increased shear and peel strength.

The wings 40 of the T-shaped diaper 10 may be made of any bonded nonwoven, such as, for example, spunbond, chemical bond, spunbond-meltblown-spunbond or hydroentangled nonwoven, that is later subjected to a water jet treatment and/or apertured. In a preferred embodiment of the invention, the nonwoven is spunbond nonwoven.

It should be appreciated that the T-shaped diaper of the previous embodiment may be altered to function similar to a conventional diaper. For example, the hook-type fasteners 46 and 48 at respective lateral ends of the front portion 22 may be eliminated, and another fastener identical to hook-type fastener 41 may be disposed at the free end of the other wing 40. With this construction, at least a portion of the outer surface of the front portion 22 of the T-shaped diaper may be made of water jet treated and/or apertured bonded nonwoven, so that the hook-type fasteners at the free ends of the wings 40 can attach to the front portion 22.

Example 1 below illustrates the advantages provided by the inventive concept of water jet treating and/or aperturing a bonded nonwoven to enhance shear and peel strengths of a fastening system.

### EXAMPLE 1

The shear and peel strengths of various hook-type fastener samples were measured using a modified version of the ASTM standardized testing procedures. The shear and peel strengths of hook-type fastener samples provided by Binder GmbH of Germany and Velcro of Manchester, N.H. were measured in relation to 33.9 gsm spunbond nonwoven, the same nonwoven subjected to aperturing with 16 pins/sq. in., and the same nonwoven subjected to aperturing with 32 pins/sq. in. Testing was also done with 20 gsm nonwoven subjected to water jet treatment, and the same nonwoven subjected to both water jet treatment and aperturing with 16 pins/sq. in. As shown in Table 1, which summarizes the experimental results, a spunbond nonwoven that is subjected to water jet treatment and/or apertured has a significantly higher peel strength and shear strength in comparison to the conventional spunbond nonwoven without any of the modifications. For example, as shown for the Binder 25445 hook sample, a spunbond nonwoven having perforations formed using a pin roller with 16 pins/sq. in. has a significantly higher peel strength and shear strength compared to the same spunbond nonwoven without any modifications. Increasing the number of pins to 32 pin/sq. in. increases the peel and shear strength of the nonwoven even further. Similarly, subjecting the spunbond nonwoven to water jet treatment significantly increases the peel and shear strength, and combining water jet treatment with perforations results in the most dramatic improvement in peel and shear strength.

TABLE 1
Hook Peel and Shear Strength Comparison
2″ long × 1″ wide Hook Samples (In a tape tab + finger lift)

| | | | | | | Binder 65445 | |
|---|---|---|---|---|---|---|---|
| | Binder 25445 (TAPE52) Hook Samples | | | | | Hook Samples | |
| | | FQN | FQN | FQN | FQN 20 gsm | | FQN |
| | | 33.9 gsm | 33.9 gsm | 20 gsm | Spunbond/ | | 33.9 gsm |
| | FQN | Spunbond | Spunbond | Spunbond/ | Water Jet | FQN | Spunbond |
| | 33.9 gsm | Nonwoven- | Nonwoven- | Water Jet | Treated | 33.9 gsm | Nonwoven- |
| | Spunbond | about 16 | about 32 | Treated | Nonwoven-about | Spunbound | about 16 |
| | Nonwoven | pins/sq in | pins/sq in | Nonwoven | 16 pins/sq in | Nonwoven | pins/sq in |
| 1st Peel | | | | | | | |
| Strength | | | | | | | |
| Load at Max Load | 142.628/ | 298.389/ | 443.046/ | 532.557/ | 613.135/ | 78.026/ | 292.842/ |
| (g/2 in) | 25.7 | 25.2 | 85.3 | 96.8 | 83.8 | 56.1 | 52.1 |
| Shear Strength | | | | | | | |
| Load at Max Load | 1094.645/ | 2924.343/ | 3325.295/ | 993.601/ | 1686.611/ | 1616.699/ | 2709.794/ |
| (g/2 in) | 569.7 | 298.9 | 420.2 | 115.5 | 303.2 | 586.9 | 462.9 |

| Binder 65445 | | | | | |
|---|---|---|---|---|---|
| Hook Samples | | Velcro USA Hook Samples | | | |
| | FQN 20 gsm | | FQN | FQN | |
| FQN 20 gsm | Spunbond/ | | 33.9 gsm | 20 gsm | FQN 20 gsm |
| Spunbond/ | Wet Jet | FQN | Spunbond | Spunbond | Spunbond/ |
| Water Jet | Treated | 33.9 gsm | Nonwoven- | Water Jet | Water Jet |
| Treated | Nonwoven-about | Spunbond | about 16 | Treated | Treated-about 16 |
| Nonwoven | 16 pins/sq in | Nonwoven | pins/sq in | Nonwoven | pins/sq in |

| | | | | | | |
|---|---|---|---|---|---|---|
| 1st Peel | | | | | | |
| Strength | | | | | | |
| Load at Max Load | 231.120/ | 530.477/ | 104.840/ | 262.006/ | 446.605/ | 673.645/ |
| (g/2 in) | 114.4 | 77.2 | 60.1 | 52.2 | 55.9 | 131.3 |
| Shear Strength | | | | | | |
| Load at Max Load | 1516.114/ | 1677.780/ | 203.931/ | 1942.152/ | 1032.605/ | 2311.901/ |
| (g/2 in) | 256.1 | 640.3 | 132.5 | 613.1 | 132.6 | 72.1 |

NOTES: All above testing was done peeling and shearing from the 2 inch length side of the hook samples.

The degree of overlap in the connection between the wings 40 (to form the belt 44) will vary with the waist size of the wearer. While a large-waisted person may have the hook-type fasteners 46 and 48 contacting respective wings 40, 40, a small-waisted person may have the hook-type fasteners 46 and 48 contacting only a single wing 40 which almost completely overlaps the other wing 40. In a conventional T-shaped brief this would present a problem in suitably locating the loop landing zones (that is, the conventional female fastening elements) along the garment-facing surface of each wing 40 if the brief were to be suitable for use by both large- and small-waisted persons. Water jet treating and/or aperturing the nonwoven wings 40 obviates the need for corresponding loop-type landing zones to mate with the hook-type fasteners 41, 46 and 48. The increased shear and peel strength of the water jet treated and/or apertured bonded nonwoven allows the hook-type fastener 41 at the free end of one of the wings 40 to be securely fastened at any point along the outer surface of the other wing 40 and the hook-type fasteners 46,48 on the front portion 22 to be securely fastened at any point along the outer surface of either wing 40. Thus, the T-shaped diaper 10 is suitable for both large-waisted and small-waisted wearers.

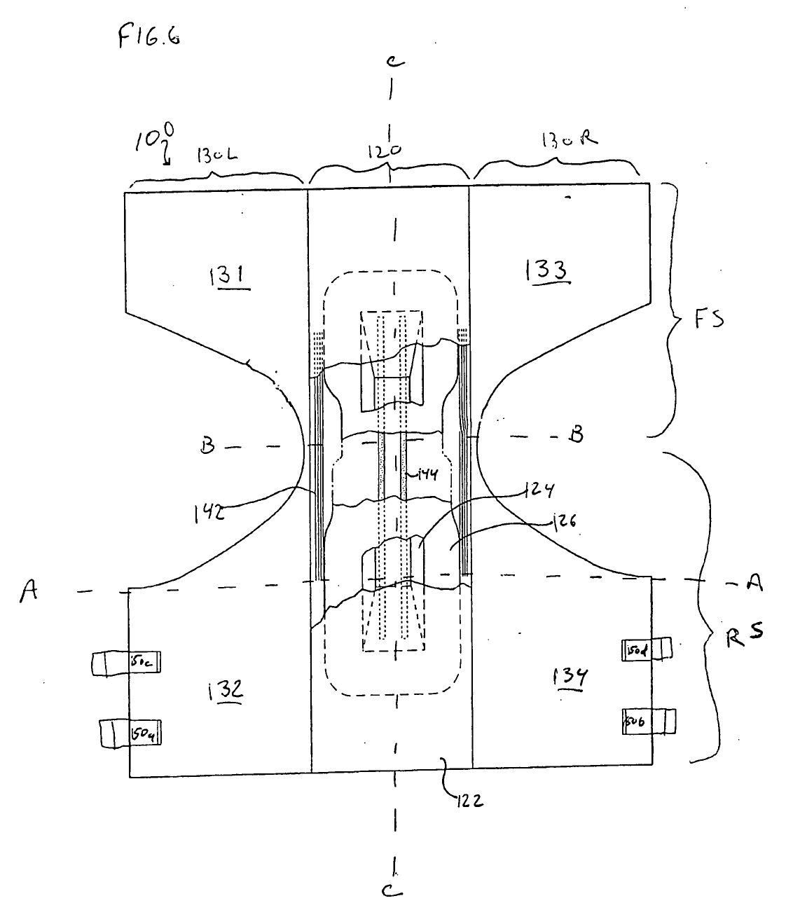

FIG. 5 is a plan view of a T-shaped diaper according to another exemplary embodiment of the invention. The T-shaped diaper according to this embodiment is the same as the previous embodiment except that the wings 40 are made of conventional bonded nonwoven, with at least one patch or landing zone 52 extending laterally across at least a portion of one of the wings 40. The landing zone 52 can be formed by water jet treating and/or aperturing at least a portion of the bonded nonwoven that is used to make the wings 40. Thus, unlike the previous embodiment, only certain portions of the wings 40 of the T-shaped diaper 10 exhibit enhanced shear and peel strength. Forming landing zones 52 by only subjecting a portion of the nonwoven fabric to water jet treatment and/or aperturing reduces the manufacturing cost of the T-shaped diaper of the present invention.

The T-shaped diaper 10 described in the previous embodiments is used to illustrate the inventive concept of using a water jet treated and/or apertured bonded nonwoven as part of an absorbent article to enhance the shear and peel strength of surfaces of the absorbent article that engage with a hook-type fastener. It should be appreciated by those skilled in the art that a bonded nonwoven subjected to water jet treatment and/or aperturing treatment is applicable to any other type of absorbent article that has a fastening system, such as conventional diapers and pull-up type absorbent articles.

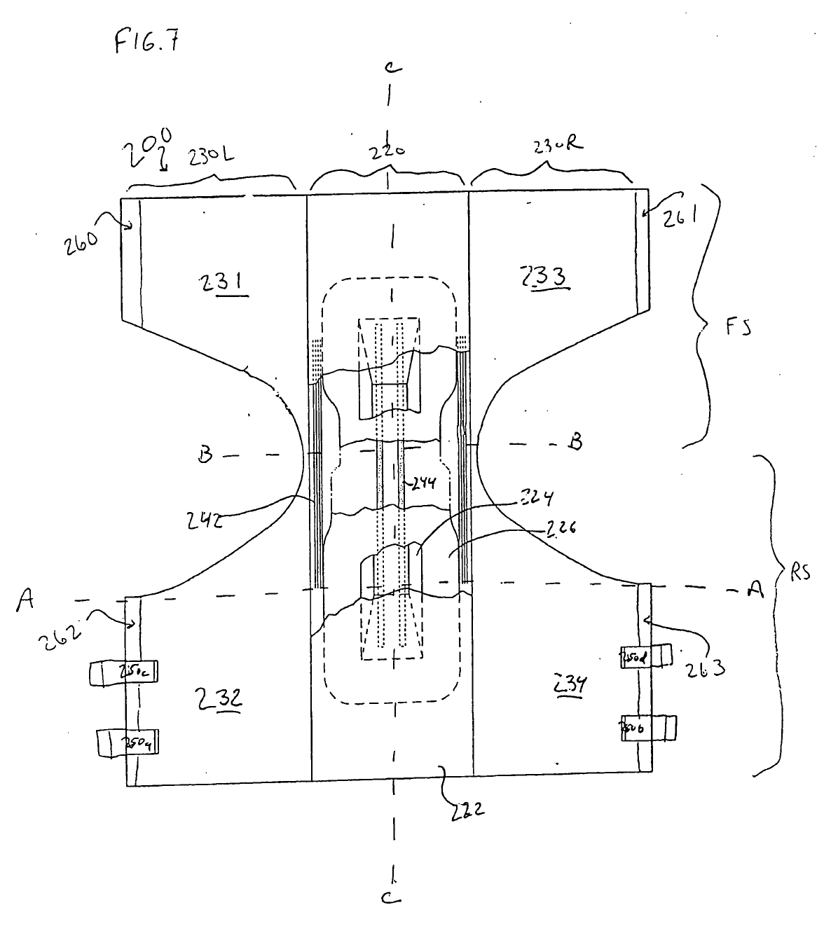

For example, FIG. 6 is a plan view of a disposable diaper according to an exemplary embodiment of the present invention that incorporates bonded nonwoven material that is water jet treated (with or without a pattern of three-dimensional relief structures) and/or apertured to increase shear and peel strength. Diaper 100 has a longitudinal axis defined by longitudinal centerline C, the term “longitudinal”, as used herein, referring to a line, axis or direction in the plane of diaper 10 that is generally aligned with, or parallel to, longitudinal centerline C and defines the length of diaper 100. Transverse axis B extends through diaper 100, intersecting longitudinal centerline C at a right angle in the plane of diaper 100, typically at about the midpoint of centerline C. Transverse axis B defines the transverse orientation relative to diaper 10 and divides diaper 10 into front and rear sections FS and RS respectively. As used herein, the term “transverse” refers to a line, axis or direction that is generally perpendicular to the longitudinal direction and defines the width of diaper 100.

The perimeter of diaper 100 is defined by backsheet 130. The diaper 100 can be divided into three regions: a containment assembly 120 which extends symmetrically along longitudinal centerline C, and two longitudinally disposed portions 130L and 130R which extend variably in the transverse direction along their length and which define the left and right sides of the diaper respectively. In its preferred embodiment, backsheet 130 of diaper 100 has an “hourglass” configuration wherein portions 130L and 130R narrow to form a crotch region at transverse axis B between front and rear sections FS and RS.

Containment assembly 120 preferably comprises a topsheet 122, an acquisition layer 124, and an absorbent core 126. Crotch elastic bands 142 and/or wetness indicators 144 may be added to one of the components of containment assembly 120. Containment assembly 120 is mounted upon backsheet 130 to form diaper 100. As shown in FIG. 6, containment assembly 120 is preferably attached to backsheet 130 symmetrically along longitudinal centerline C. As containment assembly 120 is necessarily narrower and preferably shorter than at least some portions of backsheet 130, portions of backsheet 130 are not overlapped by containment assembly 120. In the preferred embodiment, the hourglass shape of backsheet 130 results in two wings in each of longitudinal portions 130L and 130R where containment assembly 120 is not present. Wings 131 and 133 are on front section FS of the diaper 100, and wings 132 and 134 are on rear section RS of the diaper 100. Wings 131 and 133 form portions of a front waist portion, and wings 132 and 134 form portions of a back waist portion. Rear wings 132 and 134 are provided with hook-type fasteners 150a-150d. The structure of the hook-type fasters 150a-150d can be the same as the hook-type fasteners previously described for the T-shaped diaper of the present invention, and thus a further description of the hook-type fasteners is omitted here. Although there are four hook-type fasteners shown in FIG. 6 with two fasteners disposed at each wing 132 and 134, any number and configuration of hook-type fasteners can be used in conjunction with the diaper according to the various exemplary embodiments of the present invention.

At least a portion of the each of the wings 131 and 133 is made of water jet treated and/or apertured nonwoven. The wings 131 and 133 can be water jet treated with or without pillows. In at least one embodiment of the invention, the entire outer surface of the wings 131 and 133 is made of water jet treated and/or apertured nonwoven so that the hook-type fasteners 150a-150d can securely engage with any portion of the wings 131 and 133 when the diaper 100 is placed on the wearer. Thus, the fastening of the hook-type fasteners 150a-150d to the front portion of the diaper 100 is not restricted to specific landing zones. As in the wings of the previously-described T-shaped diaper, the wings 131 and 133 of the diaper 100 can be made of any single layer or multi-layer bonded nonwoven, such as, for example, a nonwoven subjected to chemical bonding, thermal bonding or hydroentanglement, that is later subjected to water jet treatment (with or without three-dimensional relief structures) and/or apertured. As discussed with reference to Table 1, a spunbond nonwoven that is water jet treated and/or apertured has a significantly higher peel strength and shear strength in comparison to the conventional bonded nonwoven without any modification.

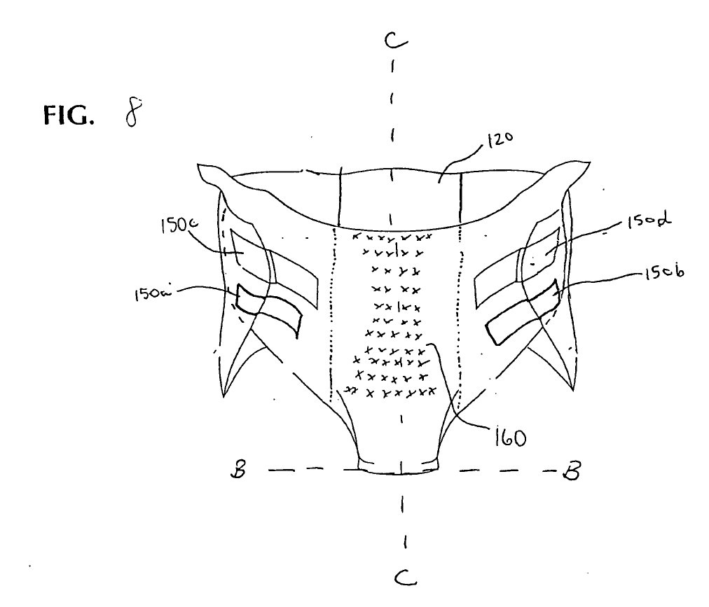

FIG. 7 is a plan view of a pull-up absorbent article according to an exemplary embodiment of the present invention, which also incorporates a bonded nonwoven that is water jet treated and/or apertured. As shown in FIG. 7, the pull-up absorbent article 200 is similar in construction to the previously described diaper of FIG. 6 except for side seals disposed at the lateral edges of the wings. As in the diaper 100, the pull-up absorbent article 200 includes a topsheet 222, a backsheet 230 and a containment assembly 220. The containment assembly 220 may include an acquisition/distribution layer 224 and an absorbent core 226. Portions of the backsheet 230 extend beyond and remain unattached from the containment assembly 220 to form an hourglass shape, resulting in two wings in each of longitudinal portions 230L and 230R. Wings 231 and 233 are on front section FS of the diaper 200, and wings 232 and 234 are on rear section RS of the diaper 100. Rear wings 232 and 234 are provided with hook-type fasteners 250a-250d. The structure of the hook-type fasters 250a-250d can be the same as the hook-type fasteners previously described, and thus a further description of the hook-type fasteners is omitted here. Although there are four hook-type fasteners shown in FIG. 7 with two fasteners disposed at each wing 232 and 234, any number and configuration of hook-type fasteners can be used in conjunction with the pull-up absorbent article according to the various exemplary embodiments of the present invention.

One of the side seals 260, 261, 262 and 263 is disposed at a lateral end of each of the wings 231-234. The side seals 260-263 can be attached to a respective wing 231-234 by, for example, an adhesive. When the wearer first puts on the pull-up type absorbent article 200, the side seal 262 is connected to the side seal 0.260 and the side seal 263 is connected to the side seal 261. Thus, the lateral end of the wing 234 is connected to the lateral end of the wing 233 and the lateral end of the wing 232 is connected to the lateral end of the wing 231, so that the pull-up type absorbent article 200 can be put on similar to conventional underwear. The side seals 260-263 are structured so as to be easily torn apart from one another when emergency situations require a rapid removal of the pull-up type absorbent article 200.

The pull-up absorbent article 200 can be replaced on the wearer by using the hook-type fasteners 250a-250d, as in the conventional diaper product. At least a portion of the each of the wings 231 and 233 at the front section FS of the pull-up type absorbent article 200 is made of water jet treated and/or apertured bonded nonwoven. If water jet treated, the bonded nonwoven can be pillowed. In at least one embodiment of the invention, the entire outer surface of the wings 231 and 233 is made of water jet treated and/or apertured bonded nonwoven so that the hook-type fasteners 250a-250d can securely engage with any portion of the wings 231 and 233 when the pull-up absorbent article 200 is placed on the wearer. Thus, the fastening of the hook-type fasteners 250a-250d to the front portion of the pull-up absorbent article 200 is not restricted to specific landing zones. As in the previously described embodiments of the invention, the wings 231 and 233 of the diaper 200 can be made of any single layer or multi-layer bonded nonwoven, such as, for example, a nonwoven subjected to chemical bonding, thermal bonding or hydroentanglement, that is later subjected to water jet treatment and/or apertured. As discussed with reference to Table 1, a spunbond nonwoven that is water jet treated and/or apertured has a significantly higher peel strength and shear strength in comparison to the conventional bonded nonwoven without any modification.

It should be appreciated that the structures of the diaper, whether conventional or T-shaped, and the pull-up type absorbent article provided herein are merely exemplary, and the present invention is meant to encompass absorbent articles having any other suitable structure that incorporates the inventive concept of a bonded nonwoven that is water jet treated and/or apertured to enhance shear and peel strength of a fastening system. For example, it should be appreciated that the backsheet may be formed of the above-identified nonwoven layered with a polypropylene or polyethylene film or as a laminate or like composite of a plurality of nonwovens.

In another exemplary embodiment, as shown in FIG. 8, the diaper 100 can be provided with at least one stay-away zone 160 at the front waist portion. The stay-away zone 160 can be formed by, for example, an inner backing film or backing film laminated to a spunbond/melt blown/spunbond nonwoven exposed through an opening in the backsheet 130. Alternatively, the entire front waist portion can be water jet treated (with or without three-dimensional relief structures) and/or apertured except for a portion of the front waist portion that forms the stay-away zone 160. Thus, the stay away zone without water jet treatment and/or aperturing will have diminished affinity to hook-type fasteners in relation to the remainder of the front waist portion.

It should be appreciated that a bonded nonwoven subjected to water jet treatment and/or aperturing as described herein may be used in any configuration in an absorbent article or in any other products that use hook-type fasteners. For example, a bonded nonwoven subjected to water jet treatment and/or aperturing may be used as landing zones in shoes/sneakers, hospital gowns, etc.

Now that the preferred embodiments of the present invention have been shown and described in detail, various modifications and improvement thereon will become readily apparent to those skilled in the art. Accordingly, the spirit and scope of the present invention is to be construed broadly and limited only by the appended claims, and not by the foregoing specification.

1. A fastening system comprising:
at least one hook-type fastener; and
a bonded nonwoven surface having at least one of perforations and three-dimensional relief structures, such that the at least one hook-type fastener is releasably attachable to the bonded nonwoven surface.

2. The fastening system of claim 1, wherein the bonded nonwoven surface has a pattern of three-dimensional relief structures.

3. The fastening system of claim 2, wherein the bonded nonwoven surface has perforations.

4. An absorbent article comprising the fastening system of claim 1.

5. The absorbent article of claim 4, further comprising:
a back waist portion having an inner surface, an outer surface, a first lateral end and a second lateral end;
a front waist portion having an inner surface, an outer surface, a first lateral end and a second lateral end; and
a crotch portion longitudinally connecting the back and front waist portions;
the at least one hook-type fastener comprising a plurality of hook-type fasteners, each of the plurality of hook-type fasteners being disposed at a corresponding one of the first and second lateral ends of the back or front waist portion, and the bonded nonwoven surface being the outer surface of the back or front waist portion.

6. The absorbent article of claim 4, further comprising:
a back waist portion having an inner surface, an outer surface, a first lateral end and a second lateral end;
a front waist portion having an inner surface, an outer surface, a first lateral end and a second lateral end;
a first wing extending from the first lateral end of the back waist portion;
a second wing extending from the second lateral end of the back waist portion; and
a crotch portion longitudinally connecting the back and front waist portions;
the at least one hook-type fastener comprising a plurality of hook-type fasteners, each of the plurality of hook-type fasteners being disposed at a corresponding one of the first and second lateral ends of the front waist portion and a lateral free end of one of the first and second wings, and the bonded nonwoven surface being the outer surface of at least one of the first and second wings.

7. An absorbent article comprising:
a topsheet;
a backsheet;
an absorbent core sandwiched between the topsheet and the backsheet;
a back waist portion having an inner surface, an outer surface, a first lateral end and a second lateral end;
a front waist portion having an inner surface, an outer surface, a first lateral end and a second lateral end;
at least one hook-type fastener disposed at each of the first and second lateral ends of the back waist portion; and
a crotch portion longitudinally connecting the back and front waist portions;
at least a portion of the outer surface of the front waist portion being made of a bonded nonwoven having at least one of perforations and three-dimensional relief structures, and the at least one hook-type fastener being releasably attachable to the at least a portion of the outer surface of the front waist portion.

8. The absorbent article of claim 7, wherein the nonwoven has a pattern of three-dimensional relief structures.

9. The absorbent article of claim 8, wherein the nonwoven has perforations.

10. The absorbent article of claim 7, wherein each of the hook-type fasteners comprises:
a first end portion attached to the back waist region; and
a second end portion extending from the corresponding one of the first and second lateral ends of the back waist portion, the second end portion comprising a hook strip.

11. The absorbent article of claim 7, wherein each of the first and second lateral ends comprises a side seal.

12. The absorbent article of claim 11, wherein each of the side seals is adhesively secured to its respective lateral end.

13. The absorbent article of claim 7, wherein at least one of the back and front waist portions is elasticated.

14. The absorbent article of claim 7, wherein the bonded nonwoven is a spunbond, chemically bonded, thermally bonded or hydroentangled nonwoven.

15. The absorbent article of claim 7, further comprising a stay-away zone formed at a portion of the front waist portion that has less affinity to accept the at least one hook-type fastener than the remainder of the front waist portion.

16. The absorbent article of claim 15, wherein the stay-away zone is formed by an opening in the backsheet that exposes a backing film.

17. The absorbent article of claim 15, wherein the entire outer surface of the front waist portion has at least one of perforations and three-dimensional relief structures except for the stay-away zone.

18. An absorbent article comprising:
a topsheet;
a backsheet;
an absorbent core sandwiched between the topsheet and the backsheet;
a back waist portion having an inner surface, an outer surface, a first lateral end and a second lateral end;
a front waist portion having an inner surface, an outer surface, a first lateral end and a second lateral end;
a first wing extending from the first lateral end of the back waist portion;
a second wing extending from the second lateral end of the back waist portion;
at least one hook-type fastener disposed at each of the first and second lateral ends of the front waist portion and at a lateral free end of one of the first and second wings;
a crotch portion longitudinally connecting the back and front waist portions;
at least a portion of the outer surface of at least one of the first and second wings being made of a bonded nonwoven having at least one of perforations and three-dimensional relief structures, the hook-type fasteners being releasably attachable to the bonded nonwoven.

19. The absorbent article of claim 18, wherein the bonded nonwoven has a pattern of three-dimensional relief structures.

20. The absorbent article of claim 19, wherein the bonded nonwoven is has perforations.

21. The absorbent article of claim 18, wherein each of the hook-type fasteners disposed at each of the first and second lateral ends of the front waist portion comprises:
a first end portion attached to the front waist portion; and
a second end portion extending from the corresponding one of the first and second lateral ends of the front waist portion, the second end portion comprising a hook strip.

22. The absorbent article of claim 18, wherein each of the hook-type fasteners disposed at the lateral free end of one of the first and second wings comprises:
a first end portion attached to the lateral free end of the one of the first and second wings; and
a second end portion extending from the lateral free end of the one of the first and second wings, the second end portion comprising a hook strip.

23. The absorbent article of claim 18, wherein at least one of the back and front waist portion is elasticated.

24. The absorbent article of claim 18, wherein the bonded nonwoven is a spunbond, chemically bonded, thermally bonded or hydroentangled nonwoven.

25. An absorbent article comprising:
a topsheet;
a backsheet defining a back waist portion and a front waist portion, the backsheet having an outer surface and an inner surface in relation to a wearer;
a containment assembly disposed between the topsheet and the backsheet;
a crotch portion disposed longitudinally between the back waist portion and the front waist portion; and
at least one hook-type fastener disposed at each lateral end of the back waist portion;
at least a portion of the outer surface of the backsheet at the front waist portion being made of a bonded nonwoven having at least one of perforations and three-dimensional relief structures, and the at least one hook-type fastener being releasably attachable to the at least a portion of the outer surface of the backsheet at the front waist portion.

26. The absorbent article of claim 25, wherein the bonded nonwoven has a pattern of three-dimensional relief structures.

27. The absorbent article of claim 26, wherein the bonded nonwoven has perforations.

28. The absorbent article of claim 25, wherein the bonded nonwoven is a spunbond, chemical bonded, thermally bonded or hydroentangled nonwoven.

29. A fastening system comprising:
at least one hook-type fastener; and
at least a portion of a bonded nonwoven surface subjected to water jet treatment and aperturing, such that the at least one hook-type fastener is releasably attachable to the bonded nonwoven surface with a peel strength in the range of about 547 g/2 in to about 663 g/2 in and a shear strength in the range of about 1595 g/2 in to about 2187 g/2 in.

30. A fastening system comprising:
at least one hook-type fastener; and
at least a portion of a bonded nonwoven surface subjected to water jet treatment, such that the at least one hook-type fastener is releasably attachable to the bonded nonwoven surface with a peel strength in the range of about 277 g/2 in to about 930 g/2 inch and a shear strength in the range of about 943 g/2 in to about 3633 g/2 in.

31. A fastening system comprising:
at least one hook-type fastener; and
at least a portion of a bonded nonwoven surface subjected to aperturing, such that the at least one hook-type fastener is releasably attachable to the bonded nonwoven surface with a peel strength in the range of about 253 g/2 in to about 393 g/2 in and a shear strength in the range of about 2222 g/2 in to about 3228 g/2 in.

32. A method of forming a female portion of a fastening system, comprising:
bonding a nonwoven fabric; and
subjecting at least a portion of the bonded nonwoven fabric to at least one of aperturing and water jet treatment.

33. The method of claim 32, wherein the bonding step comprises at least one of thermal bonding, point bonding, ultra-sonic bonding, hot-air bonding, chemical bonding, and hydro-entanglement.

34. The method of claim 32, wherein the bonded nonwoven fabric is subjected to aperturing, and the aperturing is performed by passing the bonded nonwoven fabric through a needling machine.

35. The method of claim 32, wherein the bonded nonwoven fabric is subjected to water jet treatment, and the water jet treatment is performed by subjecting a surface of the bonded nonwoven fabric to at least one row of water jets while an opposite surface of the bonded nonwoven fabric is sucked by vacuum to a forming surface.

36. The method of claim 32, wherein the bonded nonwoven fabric is subjected to water jet treatment, and the water jet treatment comprises forming three-dimensional relief structures on a surface of the bonded nonwoven fabric.

37. The method of claim 34, wherein the needling machine comprises pins in an amount within a range of about 6 to about 200 pins/sq. in.

38. The absorbent article of claim 7, wherein the nonwoven is point bonded.

39. The absorbent article of claim 16, wherein the backing film is laminated to a spunbond/melt blown/spunbond nonwoven.
